(19) United States
(12) Patent Application Publication
(10) Pub. No.: US 2003/0032921 A1
(43) Pub. Date: Feb. 13, 2003

(54) Catheter having a tapered tip

(75) Inventor: Jacky G. Duchamp, Campbell, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(73) Assignee: Advanced Cardiovascular Systems, Inc.

(21) Appl. No.: 09/924,783

(22) Filed: Aug. 7, 2001

(51) Int. Cl. A61M 29/00

(52) U.S. Cl. 604/103; 606/194

(57) ABSTRACT

A balloon catheter having a distal end, including an elongated catheter shaft having a proximal end, a distal end, a proximal shaft section, a distal shaft section, a guidewire receiving lumen extending along at least a portion thereof to a port at the catheter shaft distal end, and an inflation lumen; a balloon on the distal catheter shaft section and having an inflatable interior in fluid communication with the inflation lumen, proximal and distal ends, a proximal balloon shaft section adjacent the balloon proximal end, and a distal balloon shaft section adjacent the balloon distal end and being adhesively secured to the catheter shaft; and a tip member on the distal end of the catheter having proximal and distal ends; and being in fluid communication with the catheter shaft guidewire receiving lumen; the proximal end adhesively joined to the balloon distal shaft section and the catheter shaft.

### FIELD OF INVENTION

This invention generally relates to medical devices, and particularly to intraluminal catheters.

### BACKGROUND OF THE INVENTION

In percutaneous transluminal coronary angioplasty (PTCA) procedures, a guiding catheter is advanced until the distal tip of the guiding catheter is seated in the ostium of a desired coronary artery. A guidewire, positioned within an inner lumen of a dilatation catheter, is first advanced out of the distal end of the guiding catheter into the patient's coronary artery until the distal end of the guidewire crosses a lesion to be dilated. Then the dilatation catheter having an inflatable balloon on the distal portion thereof is advanced into the patient's coronary anatomy, over the previously introduced guidewire, until the balloon of the dilatation catheter is properly positioned across the lesion.

Once properly positioned, the dilatation balloon is inflated with liquid one or more times to a predetermined size at relatively high pressures (e.g. greater than 8 atmospheres) so that the stenosis is compressed against the arterial wall and the wall expanded to open up the passageway. Generally, the inflated diameter of the balloon is approximately the same diameter as the native diameter of the body lumen being dilated so as to complete the dilatation but not overexpand the artery wall. Substantial, uncontrolled expansion of the balloon against the vessel wall can cause trauma to the vessel wall. After the balloon is finally deflated, blood flow resumes through the dilated artery and the dilatation catheter can be removed therefrom.

In such angioplasty procedures, there may be restenosis of the artery, i.e. reformation of the arterial blockage, which necessitates either another angioplasty procedure, or some other method of repairing or strengthening the dilated area. To reduce the restenosis rate and to strengthen the dilated area, physicians frequently implant an intravascular prosthesis, generally called a stent, inside the artery at the site of the lesion. Stents are usually delivered to a desired location within a coronary artery in a contracted condition on a balloon of a catheter which is similar in many respects to a balloon angioplasty catheter, and expanded to a larger diameter by expansion of the balloon. The balloon is deflated to remove the catheter and the stent left in place within the artery at the site of the dilated lesion.

Catheters designed for intravascular procedures such as angioplasty have a number of design considerations. Such catheters must be able to transmit force along the length of the catheter shaft so that the catheter can be pushed through the patient's vasculature. However, the catheter shaft must also have sufficient flexibility to allow the catheter to track over a guidewire through tortuous vasculature as well as crossing stenosed portions of the vascular anatomy.

Prior art intravascular catheters have commonly included a soft distal tip to prevent or minimize injury to the vessel during advancement of the catheter therein. One difficulty has been forming a connection between the soft tip and the catheter which is sufficiently strong to prevent disengagement of the soft tip or kinking at the junction between the soft tip and catheter shaft. Additionally, it is necessary to balance the strength of the connection between the soft tip and the catheter shaft with the need to minimize the stiffness of the distal end of the catheter. Minimizing the stiffness of the distal end of the catheter results in improved maneuverability of the catheter.

Accordingly, it would be a significant advance to provide a catheter with a soft tip having improved performance. This invention satisfies these and other needs.

### SUMMARY OF THE INVENTION

The present invention is directed to balloon catheter with improved maneuverability. The catheter includes an elongated catheter shaft having a proximal end, a distal end, and proximal and distal shaft sections. A guidewire receiving lumen extends along at least a portion of the catheter shaft to a port at the distal end of the catheter shaft. An inflation lumen extends along at least a portion of the catheter shaft terminating at a point proximal to the distal end of the catheter shaft.

An inflatable member, such as a balloon, with proximal and distal ends and an inflatable interior is disposed on the distal section of the catheter shaft. The interior of the balloon is in fluid communication with the inflation lumen. The balloon further includes a distal shaft section adjacent the balloon distal end. The distal end of the balloon is adhesively secured to the catheter shaft. The balloon distal shaft section tapers distally and forms a tapered balloon distal end with an interior surface defining a portion of the guidewire receiving lumen.

The catheter further includes a tip member on the distal end of the catheter having proximal and distal ends and being in fluid communication with the catheter shaft guidewire receiving lumen. The proximal end of the tip member is adhesively joined to the balloon distal shaft section and the catheter shaft.

In one embodiment, the proximal end of the tip member extends proximally over the distal end of the catheter shaft forming a butt-joint with the distal end of the balloon distal shaft section.

A layer of adhesive, preferably uv-cured adhesive, extends along the length of the catheter shaft extending underneath the balloon distal shaft and the distal tip member.

In a method of making the catheter of the present invention having the distal tip portion, a catheter assembly is provided including a catheter shaft having proximal and distal ends, and a balloon having proximal and distal ends with an inflatable interior and a distal shaft section with an interior surface. A tip member having proximal and distal ends is further provided. The distal end of the catheter shaft is positioned within the interior of the balloon distal shaft section and terminates at a point distal to the balloon distal end. Adhesive is present along the exterior surface of the catheter shaft extending underneath the balloon distal shaft. The proximal end of the tip member is positioned adjacent the balloon distal end. The adhesive is cured, preferably uv-cured, thus bonding at least a portion of the balloon distal shaft section to the catheter shaft and bonding at least a portion of the balloon distal shaft section to the tip member and forming the distal tip portion of the catheter.

These and other advantages of the invention will become more apparent from the following detailed description when taken in conjunction with the accompanying exemplary drawings.

### BRIEF DESCRIPTION OF THE DRAWINGS

FIG. 1 is an elevational view, partially in section, of a balloon catheter embodying features of the invention, having a tapered distal tip member.

FIG. 2 is an enlarged longitudinal cross sectional, partially cutaway, view of the catheter of FIG. 1.

FIG. 3 is a transverse cross-section of the catheter of FIG. 2 taken along lines 3-3.

FIGS. 4 is a transverse cross-section of the catheter of FIG. 2 taken along lines 4-4.

FIGS. 5 is a transverse cross-section of the catheter of FIG. 2 taken along lines 5-5.

FIGS. 6A through 6D illustrate an embodiment of a method of making the catheter of the present invention.

### DETAILED DESCRIPTION OF THE INVENTION

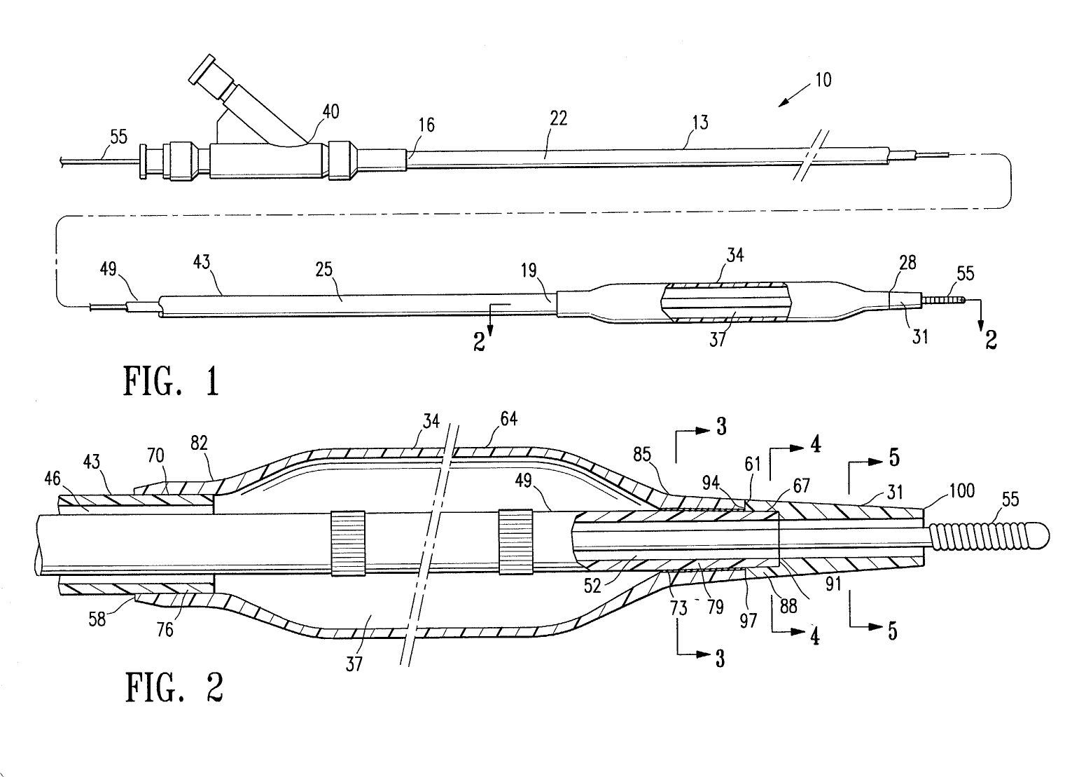

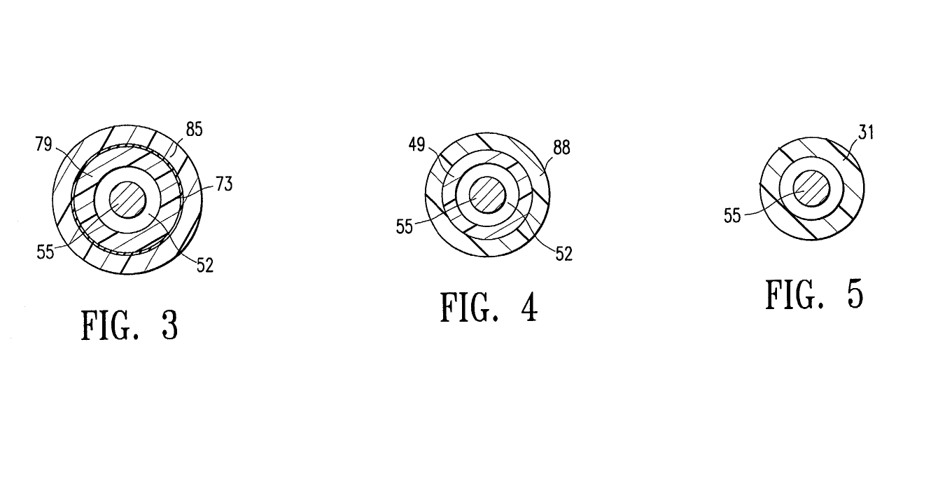

FIGS. 1 through 5 illustrate a balloon catheter 10 embodying features of the invention, comprising an elongated catheter shaft 13 having proximal and distal ends 16 and 19, proximal and distal shaft portions 22 and 25, a distal tip portion 28 including a tip member 31 on the catheter shaft distal end 19, an inflatable balloon 34 on the distal catheter shaft portion 25 having an interior 37, and an adapter 40 on the proximal catheter shaft portion 22 for directing inflation fluid, among other things, to and from the catheter 10.

In the embodiment illustrated in FIG. 1, the catheter shaft distal portion 25 comprises an outer tubular member 43 having an inflation lumen 46, and an inner tubular member 49 having a guidewire receiving lumen 52 disposed within the inflation lumen 46 for slidably receiving a guidewire 55. Balloon 34 has proximal and distal ends 58 and 61 and an intermediate section 64 disposed therebetween.

The inner tubular member 49 includes an inner member extension length 67 extending distally beyond the balloon distal end 61.

The Balloon 34 is sealingly secured, at proximal and distal seals 70 and 73, to a distal portion 76 of the outer tubular member 43 and a distal portion 79 of the inner tubular member 49, at balloon proximal and distal shaft sections 82 and 85, respectively. In one embodiment, the distal seal 73 extends along the entire length of the balloon distal shaft section 85. The balloon interior 37 is in fluid communication with the inflation lumen 46 and the adapter 40. The distal seal 73, preferably has a longitudinal dimension ranging from about 0.25 to about 1.0 millimeters (mm), more preferably, ranging from about 0.5 to about 0.75 (mm).

A proximal portion 88 of the tip member 31 extends proximally over a distal end 91 of the inner member extension length 67, and forms a butt-joint 94 with the balloon distal end 61.

The proximal balloon seal, the distal balloon seal 73, and the butt-joint 94 are formed by any suitable means such as heat or adhesive bond. The distal seal and the butt-joint, are preferably, formed by adhesive bond, with the adhesive, preferably, extending along the entire length of the balloon distal shaft.

The distal tip portion 28 defines in part the guidewire lumen 52. The tip member 31 is, preferably, tapered in the distal direction. The tip member 31, preferably, is extruded in a tapered fashion, preferably having a constant thickness and a constant taper angle. However, the tip member 31 can be tapered using any other suitable means such as laser heat treatment with the aid from a tapered mandrel. In one embodiment, a proximal outer diameter of the tip member 31 at a tip member proximal base 97 ranges from about 0.025 to about 0.028 inches, preferably from about 0.025 to about 0.026 inch; to a distal outer diameter of tip member 31 at a tip member distal base 100 ranging from about 0.019 to about 0.018 inch, preferably from about 0.018 to about 0.017 inch. The tip member 31, preferably has a thickness ranging from about 0.0025 to about 0.005 inch, preferably, from about 0.003 to about 0.004 inch.

The inner member extension length 67, preferably has a longitudinal dimension ranging from about 1.0 to about 0.75 (mm), preferably ranging from about 0.75 to about 0.5 mm, typically 0.5 mm. The proximal portion 88 of the tip member 31 extending over the inner member extension length 67, has a longitudinal dimension ranging from about 0.5 to about 0.25 mm, preferably ranging from about 0.35 to about 0.25 mm.

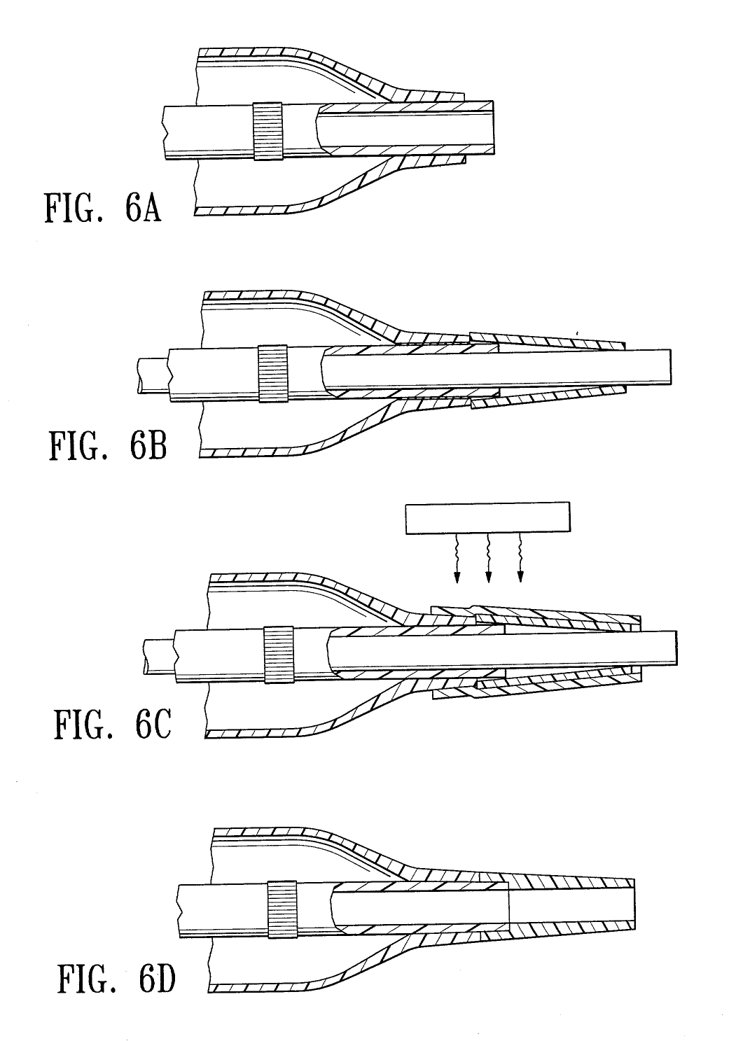

FIGS. 6A through 6D illustrate features of one presently preferred embodiment of a method of making the catheter of the present invention. The catheter of the present invention is preferably made by aligning the balloon and the inner member at desired locations. Adhesive is inserted into the gap area formed between an inner surface of the balloon distal shaft and an outer surface of the inner tubular member, preferably, along the entire length, preferably, along the entire inner surface, of the balloon distal shaft. The tip member is then extended over the distal portion of the inner member extending beyond the balloon distal end in abutting relation to the balloon distal end.

A mandrel 200 is positioned in the catheter, preferably, terminating distally beyond the inner tubular member distal end. The adhesive area is then cured ( e.g. UV curable adhesive), as by exposure to a UV source, forming a seal between the balloon distal shaft and the distal portion of the inner member and a butt-joint between the balloon distal end and the tip member proximal end.

The dimensions of catheter 10 are determined largely by the size of the artery or other body lumen through which the catheter must pass or the size of the stent being delivered. Typically, the outer tubular member 46 has an outer diameter of about 0.02 to about 0.04 inch (0.05 to 0.10 cm), usually about 0.037 inch (0.094 cm), an inner diameter of about 0.015 to about 0.035 inch (0.038 to 0.089 cm), usually about 0.03 inch (0.076 cm). The wall thickness of the outer tubular member 46 can vary from about 0.002 to about 0.008 inch (0.0051 to 0.0201 cm), typically about 0.003 inch (0.0076 cm). The inner tubular member 49 typically has an outer diameter of about 0.019 to about 0.028 inch, usually about 0.021 inch. The overall working length of the catheter 10 may range from about 100 to about 150 centimeters (cm), and is typically about 147 cm. Preferably, balloon 34 may have a length about 0.5 cm to about 4 cm and typically about 2 cm with an inflated working diameter of about 1 to about 8 mm, and for coronary applications about 1.5 mm to about 5 mm. The balloon has a thickness ranging from about 0.002 to about 0.0015 inch, more preferably, from about 0.015 to about 0.001 inch.

The various catheter components can be formed of suitable materials. The tubular members (e.g., inner tubular member, outer tubular member, tip member) are formed of material, or include material thereon, compatible with the balloon material to allow formation of appropriate joints therebetween.

In a presently preferred embodiment, the tip member 31 is formed of a polymeric material similar to or different from the material forming the balloon 34. The tip member 31 may be a soft tip configured to provide an atraumatic distal end on the catheter to minimize injury to the patient's vasculature during advancement of the catheter therein. In one embodiment, the tip member 31 is formed of a polymeric material similar to that forming the balloon 34 but having a lower Shore Durometer hardness than the polymeric material forming a section of the balloon proximal thereto. For example, the balloon material may be selected from material with hardness 60 and above, more preferably from about 63 to about 72; with the tip member 31 formed of a material having a hardness of 65 and below, more preferably from about 63 to about 55, on a Shore D scale.

A variety of polymeric materials may be used to form the tip member 31 including polyamides such as PEBAX (polyether block amide) and polyethylene based adhesives such as PRIMACOR, high density polyethylene (HDPE), polyurethane, and polyesters such as HYTREL. However, the choice of material depends on a variety of factors including the desired application and the method used to make the tip member 31.

To the extent not discussed herein, the various catheter components can be formed of conventional materials. Outer tubular member 46 and the inner tubular member 49 can be formed by conventional techniques, for example by extruding, from materials already found useful in intravascular catheters such a polyethylene, polyvinyl chloride, polyesters, polyamides, polyimides and composite materials. The various components may be joined by heat bonding or use of adhesives.

A variety of suitable catheter designs may be used, including rapid exchange, over-the-wire, and fixed wire catheter designs. A rapid exchange catheter generally includes an inflation lumen extending from the proximal end of the catheter shaft to a location spaced proximal to the distal end of the catheter shaft, a distal guidewire port in the distal end of the catheter shaft, a proximal guidewire port spaced distal to the proximal end of the catheter shaft, and a guidewire lumen extending between the proximal and distal guidewire ports. Typically, the proximal guidewire port is spaced a substantial distance from the proximal end of the catheter shaft and a relatively short distance from the distal guidewire port, so that the proximal guidewire port is closer to the distal guidewire port than to the proximal end of the catheter shaft.

Although not illustrated, the balloon catheter of the invention may be used to deliver prostheses, such as expandable stents, grafts, and the like, to a desired location within the patient's vasculature. A stent (not shown) comprising an expandable tubular body, typically having an open-walled structure, may be mounted on balloon 34, and balloon 34 may be inflated to expand the stent and seat it in the vessel. Additionally, catheter 10 may be used to touch up a previously implanted stent by positioning balloon within stent lumen and expanding the balloon to further expand the stent within a body lumen.

While particular forms of the invention have been illustrated and described, it will be apparent that various modifications can be made without departing from the spirit and scope of the invention. Accordingly, it is not intended that the invention be limited, except as by the appended claims.

What is claimed is:

1. A balloon catheter having a distal end, comprising:
an elongated catheter shaft having a proximal end, a distal end, a proximal shaft section, a distal shaft section, a guidewire receiving lumen extending along at least a portion thereof to a port at the catheter shaft distal end, and an inflation lumen;
a balloon on the distal catheter shaft section and having an inflatable interior in fluid communication with the inflation lumen, proximal and distal ends, a proximal balloon shaft section adjacent the balloon proximal end, and a distal balloon shaft section adjacent the balloon distal end and being adhesively secured to the catheter shaft; and
a tip member on the distal end of the catheter having proximal and distal ends and being in fluid communication with the catheter shaft guidewire receiving lumen; the proximal end adhesively joined to the balloon distal shaft section and the catheter shaft.

2. The catheter of claim 1 wherein the catheter shaft extends distally beyond the balloon distal end.

3. The catheter of claim 2 wherein the tip member proximal end forms a butt-joint with the balloon distal shaft section.

4. The catheter of claim 3 wherein the tip member proximal ends extends proximally over the distal end of the catheter shaft.

5. The catheter of claim 2 wherein the distal balloon shaft forms a lap-joint with the proximal end of the tip member.

6. The catheter of claim 2 wherein the distal end of the catheter shaft extends distally beyond the balloon distal end in a range from about 1.0 to about 5.0 millimeters.

7. The catheter of claim 6 wherein the distal end of the catheter shaft extends distally beyond the balloon distal end in a range from about 1.0 to about 5.0 millimeters.

8. The catheter of claim 4 wherein the proximal end of the tip member extends distally over the catheter shaft in a range from about 0.1 to about 0.5 millimeters.

9. The catheter of claim 8 wherein the proximal end of the tip member extends distally over the catheter shaft in a range from about 0.1 to about 0.5 millimeters.

10. The balloon catheter of claim 1 wherein the catheter shaft comprises an outer tubular member defining the inflation lumen and an inner tubular member disposed within at least a portion of the outer tubular member and defining at least in part the guidewire receiving lumen, the inner tubular member having a distal end extending through the balloon interior and extending distal to the balloon distal end.

11. The catheter of claim 1 wherein the adhesive for forming the adhesive seal between the balloon distal shaft section and the catheter shaft extends along the length of the balloon distal shaft section.

12. The catheter of claim 2 wherein the adhesive for forming the adhesive seal between the catheter shaft and the balloon distal shaft section and catheter shaft section and the tip member

13. A method of forming a distal tip portion of a balloon catheter, comprising:
providing a catheter assembly including a catheter shaft having proximal and distal ends, and a balloon having proximal and distal ends with an inflatable interior and a distal shaft section with an interior surface;
providing a tip member having proximal and distal ends;
positioning the distal end of the catheter shaft within the interior of the balloon distal shaft section and terminating at a point distal to the balloon distal end;
providing adhesive along the exterior surface of the catheter shaft extending underneath the balloon distal shaft;
positioning the proximal end of the tip member adjacent the balloon distal end;
bonding at least a portion of the balloon distal shaft section to the catheter shaft;
bonding at least a portion of the balloon distal shaft section to the tip member; and
forming the distal tip portion of the catheter.

14. The method of claim 13 further including curing the adhesive.
